(19) United States
(12) Patent Application Publication
(10) Pub. No.: US 2003/0216700 A1
(43) Pub. Date: Nov. 20, 2003

(54) Occlusion resistant medical catheter with flexible core

(75) Inventors: Robert Mark Pearson, San Jose, CA (US); Mary M. Morris, Mounds View, MN (US); Douglas Owen Hankner, Fridley, MN (US)

Correspondence Address: BANNER & WITCOFF, LTD., TEN SOUTH WACKER DRIVE, SUITE 3000, CHICAGO, IL, 60606, US

(73) Assignee: Medtronic, Inc., Minneapolis, MN

(21) Appl. No.: 10/463,687

(22) Filed: Jun. 17, 2003

Related U.S. Application Data
(62) Division of application No. 09/560,942, filed on Apr. 28, 2000.

(51) Int. Cl. A61M 25/00

(52) U.S. Cl. 604/266

(57) ABSTRACT

A medical catheter comprising a flexible tube having an outer wall and an inner wall, the inner wall defining a lumen, the lumen extending longitudinally along the flexible tube and having a cross-sectional area; and a flexible core within the lumen substantially extending longitudinally along the flexible tube, the flexible core having a cross-sectional area smaller than the cross-sectional area of the lumen at any given longitudinal point along the flexible tube and flexible core, the flexible tube and the flexible core defining a passageway, and which in combination provides structure resistant to occlusion of the passageway.

### RELATED APPLICATION

This is a divisional application of U.S. Ser. No. 09/560,942 filed Apr. 28, 2000, which is incorporated in its entirety herein by reference.

### FIELD OF INVENTION

The present invention relates to medical catheters and, in particular, to the prevention of occlusion of medical catheters. The present invention also relates to methods of using and making such medical catheters.

### BACKGROUND OF THE INVENTION

Catheters are commonly known in the medical field. Catheters are used for fluid transfer, including the delivery of drugs to various parts of the body. In many cases, it is important that the fluid flow not be interrupted. Various conditions and procedures can result in the occlusion of the catheter resulting in the stoppage of fluid flow. For example, conventional catheters can kink, i.e., double over itself like a garden hose, resulting in occlusion. Conventional catheters have been designed to prevent this occlusion by structures that are resistant to kinking of the catheters. One common approach has been to thicken and strengthen the wall of the catheter by adding strengthening materials within the catheter wall to prevent the catheter from kinking. While this approach may resist kinking, it also presents numerous disadvantages. For example, by thickening the wall of the catheter, valuable space is taken away from the area used for fluid flow, thus reducing the amount of the flow. In addition, a stiff catheter may cause damage to tissue surrounding the catheter.

Another approach to prevent kinking is to add a separate stiffening member. For example, U.S. Pat. No. 5,269,752 discloses a stiffening member in the lumen of the catheter. The stiffening member prevents the catheter from kinking into such a tight radius that would cause occlusion. However, in this structure the stiffening member does not permit the catheter to flex, and thus its use for catheters is limited, and cannot be used in circumstances that require a flexible catheter.

An important characteristic of catheters is radiopaqueness, which is the ability of the catheter to be visualized by X-ray or fluoroscopy. Radiopaqueness of a medical component, such as a catheter, depends upon the type of material that the component is made from. Barium sulfate is a material that is radiopaque, and has been used in conventional catheter walls. However, when barium sulfate is added to the wall of catheter, it reduces the mechanical properties of the catheter significantly. Thus, a catheter containing barium sulfate generally has a thick outer wall, which reduces the cross-sectional area for fluid flow.

Some conventional catheters use a heavy metal, such as tungsten, tantalum, gold, or platinum, instead of barium sulfate in the catheter wall, e.g. as a radiopaque ring for marking. However, in these conventional catheters, the heavy metal is in direct contact with patient tissue. It would be more preferable that heavy metal not is in direct contact with patient tissue for biocompatibility purposes. Further, it is preferable that such radiopaque material is not in the catheter wall since its presence can effect the mechanical properties of the catheter wall. Some other conventional devices, like implantable pacing devices, have used tungsten as a radiopaque material for lettering to identify the type of implanted device and which can be read via X-ray or fluoroscopy, but do not use such radiopaque material for catheter marking.

Thus, there exists a need for a catheter that prevents occlusion, but allows for bending and curving of the catheter. Further, there is a need for a catheter that is radiopaque yet does not have the radiopaque material in direct contact with patient tissue.

### SUMMARY OF THE INVENTION

A catheter has now been invented that overcomes the deficiencies and disadvantages of conventional catheters. One preferred embodiment of the present invention is a reinforced medical catheter for fluid transfer (e.g. drug delivery) comprising a flexible tube having both an inlet opening and outlet opening, the inner wall defining a lumen that extends longitudinally along the flexible tube which has a cross-section and a flexible core positioned within the lumen and which substantially extends longitudinally along the flexible tube. The flexible core has a cross-sectional area smaller than the cross-sectional area of the lumen at any given longitudinal point along the flexible tube and flexible core. Thus, the flexible tube and the flexible core define a fluid passageway. In a preferred embodiment, the cross-sectional area of the flexible tube may be substantially constant.

The flexible core is made of any suitable material that can flex, but cannot be substantially compressed. Since the flexible core has a substantially constant cross-sectional area smaller than the cross-sectional area of the lumen at any given point along the flexible tube and flexible core, there will always be a fluid passageway by the flexible tube and flexible core at any given longitudinal point. Further, because of this unique construction, the ratio of the major and minor axes of the flexible tube at any given longitudinal point along the flexible tube does not vary greatly, and highly elliptical shapes of the flexible tube from crimping or quashing of the flexible tube are prevented.

In addition, because of the unique construction of the present invention, a heavy metal, such as tantalum or tungsten, can be used in the flexible core to provide radiopaqueness at a site other than the catheter wall, thereby eliminating the need for enlarging the thickness of the catheter wall to account for the reduction in mechanical properties due to the presence of barium sulfate in the catheter wall in conventional catheters.

It is an object of the present invention in one embodiment to prevent occlusion of a flexible catheter by maintaining a fluid passageway, even after kinking or pinching of the catheter has occurred.

Another object of the invention in one embodiment is to change the stiffness of the distal end of the catheter by changing the stiffness of the material or dimensions of the material at the distal end of the catheter.

Another object of the invention in one embodiment is to eliminate the need for a styletguide wire, which is necessary for insertion and placement of conventional catheters.

Another object of the invention in one embodiment to provide a method of manufacturing the catheters of the present invention via co-extrusion of the flexible tube and flexible core. Another object of the invention in one embodiment is to provide a catheter with greater radiopaqueness and in a component that is not in direct contact with bodily tissue, while maintaining a thin walled catheter and allowing for the maximum amount of fluid flow through the catheter.

### BRIEF DESCRIPTION OF THE DRAWINGS

FIG. 1A and FIG. 1B illustrate preferred embodiments of the catheter of the present invention when used in connection with a medical device 21.

FIG. 2 is a cross-section of a preferred embodiment of the catheter of the present invention.

FIG. 3 is a cross-section of the preferred embodiment illustrated in FIGS. 1 and 2, taken from the distal end of the catheter of the present invention.

FIG. 4 is a cross-section of an alternative embodiment of the catheter of the present invention, taken from the distal end of the catheter, and wherein the flexible core is attached to the inner wall of the catheter.

FIGS. 5A, 5B, and 5C are perspective cross sections that illustrate alternative cross-sectional shapes for the flexible core of the present invention.

FIG. 6 is a perspective cross-section that illustrates an alternative embodiment of the flexible core and flexible tube of the catheter of the present invention.

FIG. 7 is a cross-section of an alternative embodiment of the catheter of the present invention shown in FIG. 3, wherein the flexible core 9 has a jacket 40.

FIG. 8 is a perspective cross-section that illustrates an alternative embodiment wherein the flexible core 9 comprises a wire bundle 60 and a jacket 40.

### DESCRIPTION OF THE PREFERRED EMBODIMENTS

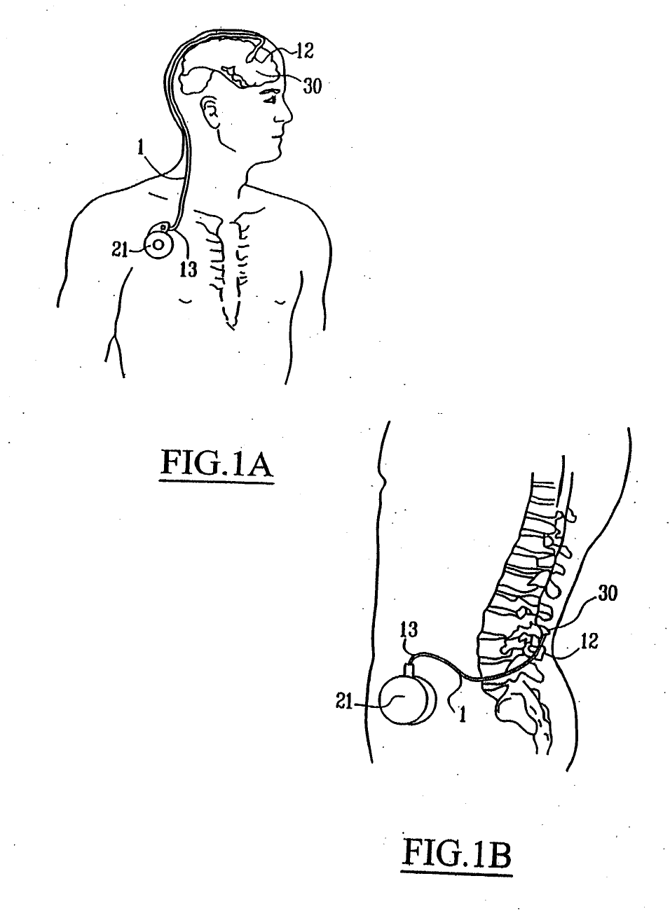

In FIGS. 1A, 1B, 2, and 3, the medical catheter 1 of a preferred embodiment of the present invention is illustrated. As shown in FIGS. 1A, 1B, 2, and 3, the medical catheter 1 comprises a flexible tube 2 having an inlet opening 3 at proximal end 13, and at least one outlet hole 4 near distal end 12. Distal end 12 is in contact with patient tissue 30. The flexible tube 2 has an outer wall 5 and an inner wall 6. The inner wall 6 defines a lumen 7, which extends longitudinally along the flexible tube 2 and which has a cross-section 8.

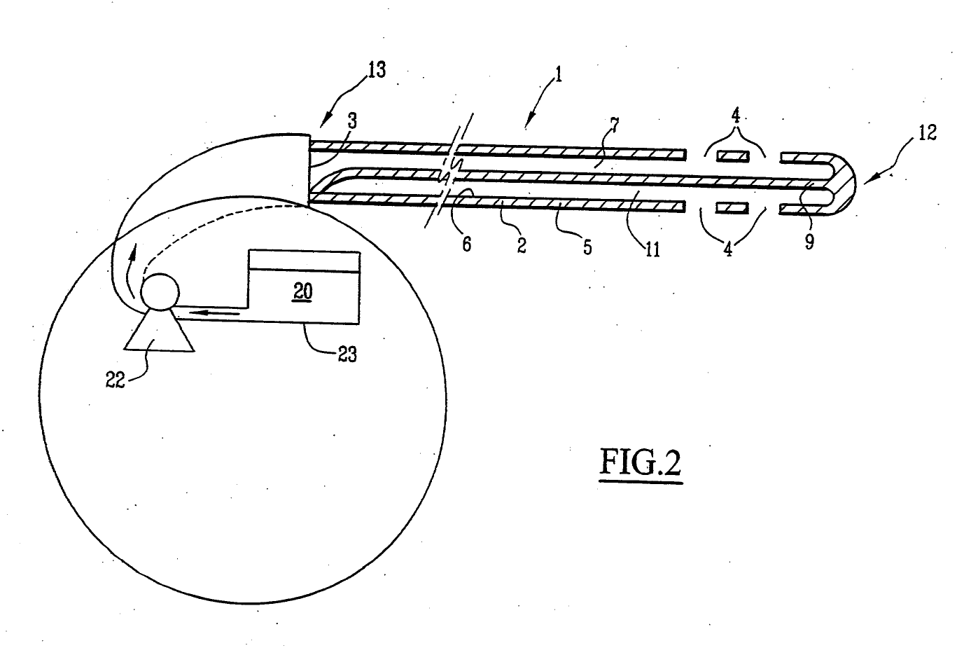

A flexible core 9 is within the lumen 7 and substantially extends longitudinally along the flexible tube 2. The flexible core 9 preferably has a substantially constant cross-section 10 smaller than the cross-section of the lumen 8 at any given longitudinal point along the flexible tube 2 and flexible core 9. The flexible tube 2 and the flexible core 9 define a fluid passageway 11. Fluid passageway 11 can be a drug passageway for the delivery of a drug 20 from drug delivery device 21 via pump 22. Drug 20 can be supplied to pump 22 from reservoir 23. As shown in FIG. 2, in a preferred embodiment, flexible core 9 is attached to distal end 12 and proximal end 13.

While FIGS. 1A and 1B show the distal end at or near brain or spinal tissue respectively, the present invention can also be used in vascular treatment, e.g., where the distal end is placed at or near vascular tissue or other sites common to catheters as will be clear to those skilled in the art.

As shown in FIG. 4, an alternative preferred embodiment of the invention has the flexible core 9 attached to the inner wall 6 of the lumen 7. The flexible core 9 can either be permanently attached to the inner wall 6 of the lumen 7 or the flexible core 9 can be free to break away to move freely within the lumen 7.

The stiffness of the flexible tube 2 can be varied (such as choice of flexible tube material 15 and/or wall thickness), and thus the need for a stylet guide wire, which is required for the insertion and placement of conventional catheters, is eliminated. Further, by eliminating the stylet guide wire, there is space within lumen 7 for insertion of flexible core 9. Because the need for the stylet guide wire is eliminated by the present invention, there is no need for medical personnel to remove such stylet guide wires after insertion and placement of the catheter, or for the patient to be effected or inconvenienced by such removal and/or for the catheter to move from the desired site when the stylet guide wire is removed.

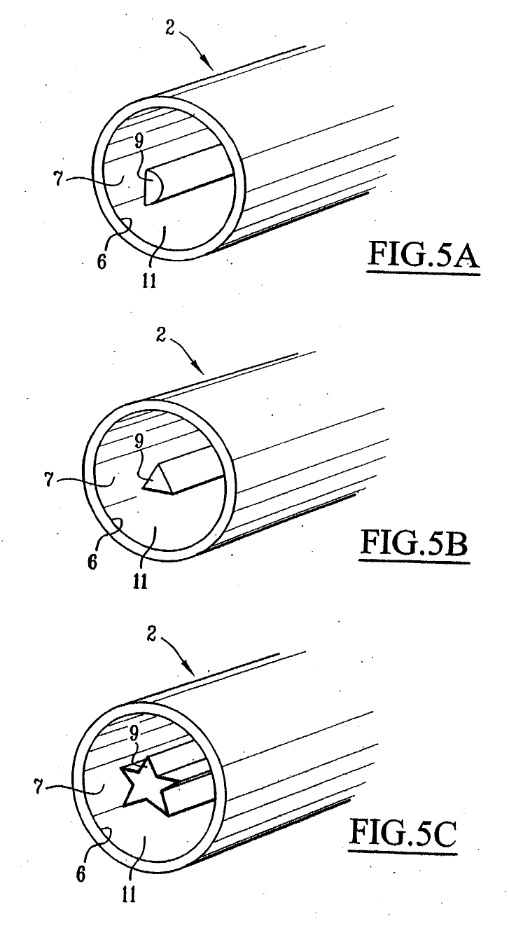

As shown in FIGS. 5A, 5B, and 5C, alternative preferred embodiments include various shapes of the flexible core 9. A flexible core 9 with a simple shape, e.g., a circle or an oval as shown in FIGS. 1-3, will allow for the greatest fluid flow. A more complex shape, like those shown in FIGS. 5A, 5B, and 5C, will lessen fluid flow within the catheter 1 because it will lessen the cross-sectional area of the passageway 11, but a more complex shape may be better at preventing occlusion, provided that the shape of the catheter tube at the same longitudinal point is different from the shape of flexible core 9. FIG. 5A shows a “D” shaped flexible core 9 within lumen 7 defined by inner wall 6 of flexible tube 2. FIG. 5B shows a triangle shaped flexible core 9 within lumen 7 defined by inner wall 6 of flexible tube 2. FIG. 5C shows a star shaped flexible core 9 within lumen 7 defined by inner wall 6 of flexible tube 2. Other shapes for flexible core 9 will occur to those skilled in the art.

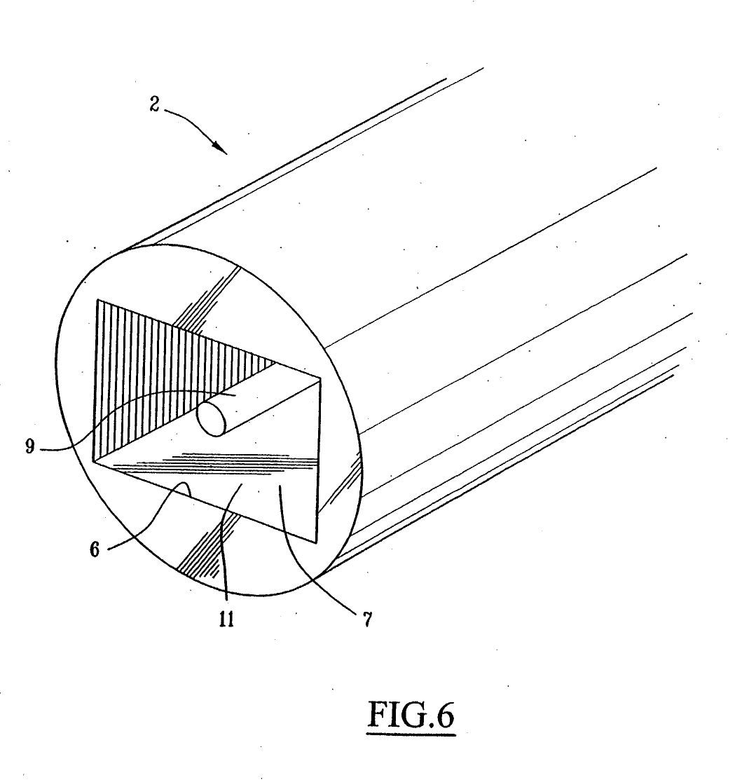

FIG. 6 shows an alternative preferred embodiment of the present invention wherein the outer shape of flexible tube 2 is circular, the cross-sectional area of lumen 7 has a non-circular shape, and flexible core 9 has a cross-sectional area having a circular shape. Although lumen 7 as shown is rectangular in cross-sectional area, lumen 7 may have any non-circular cross-sectional area. Flexible core 9 is within lumen 7 defined by inner wall 6 of flexible tube 2. Passageway 11 is defined by flexible tube 2 and flexible core 9. Since the cross-sectional area of lumen 7 is non-circular in shape and the cross-sectional area of flexible core 9 is circular in shape, the combined structure provides greater resistance to occlusion of passageway 11 than if the cross-sectional area of lumen 7 and flexible core 9 were similar in shape.

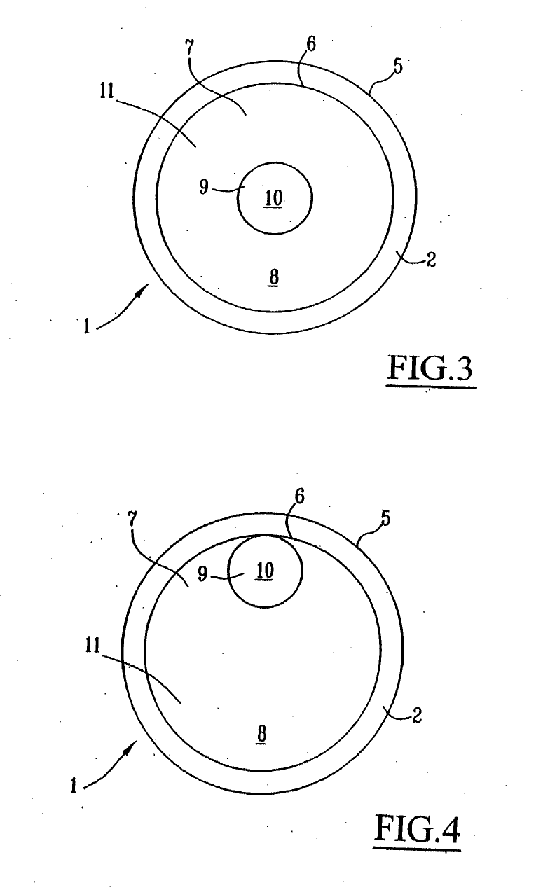

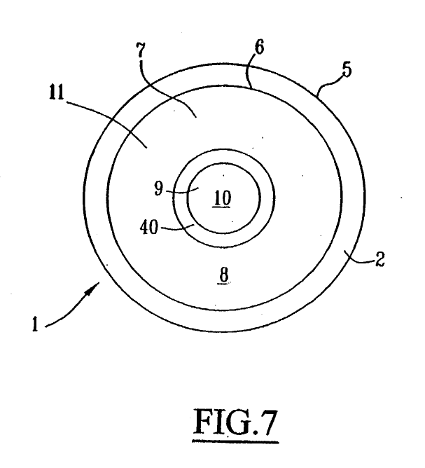

FIG. 7 is a cross-section of an alternative embodiment of the catheter of the present invention shown in FIG. 3, wherein the flexible core 9 has a jacket 40. Jacket 40 can comprise any suitable material, for example, polyurethane. Flexible core 9 can be made of any suitable radiopaque core material (e.g., such as tantalum, tungsten, gold, platinum, iridium, silver, nickel, and alloys thereof). In a more preferred embodiment, flexible tube 2 will have an outside diameter of about 0.050 inches and an inside diameter of about 0.035 inches, and jacket 40 will have an outside diameter of about 0.018 inches and an inside diameter of about 0.014 inches over flexible core 9 having an outside diameter of about 0.014 inches.

The flexible tube 2 of the medical catheter 1 can be made of any suitable material. Further, the flexible core 9 can be made of any radiopaque material. Preferably, the flexible tube 2 would be made of polyurethane. Flexible core 9 can preferably be made of a heavy metal and polyurethane. Preferably, for flexible core 9, metal powder is mixed with polyurethane and the, mixture is extruded. In addition, for the embodiment shown in FIG. 7, flexible core 9 and jacket 40 can be co-extruded. Further, flexible core 9 and flexible tube 2 can be co-extruded (see e.g., the embodiment shown in FIG. 4).

Any suitable heavy metal is contemplated in accordance with the invention, including any one or more of the following: tantalum, tungsten, gold, platinum, iridium, silver, nickel, and alloys thereof. In a more preferred embodiment, tantalum is used in flexible core 9. Preferably the flexible core 9 contains up to about 95% by weight heavy metal, and more preferably up to about 80-85% by weight heavy metal. A lower amount of metal and a greater amount of polymer in flexible core 9 may provide less stress fatigue. Further, it may be desirable to have a lower amount of metal and a greater amount of polymer in flexible core 9 at the distal end 12 to provide softer physiologic contact with patient tissue. In the preferred embodiment, only the flexible core 9 would contain a radiopaque material.

Further, the flexible core 9 can have a first longitudinal portion having radiopaque material and a second portion that does not contain radiopaque material in series with the first longitudinal portion so that when viewed via X-ray or fluoroscopy, the appearance of flexible core 9 will show only the radiopaque first longitudinal portion and this information can be used for non-invasive length measurement and identification of catheter placement. This embodiment also reduces radiopaque distortion of magnetic resonance imaging (MRI). This feature may be of particular value when using MRI at distal end 12.

Alternatively, the cross-sectional shape of a first longitudinal portion of flexible core 9 can have a cross-sectional shape or size different from a second longitudinal portion of the flexible core 9 and which is in series with the first longitudinal portion. These different shapes between the first and second longitudinal portions can be made by twisting the flexible core or forming a bead in flexible core 9 having a different shape and/or size than another portion of flexible core 9. The number and positioning of twists and/or beads will advise the viewer of catheter 1 via X-ray or fluoroscopy of the positioning of the catheter within the patient and provide visual assistance for insertion and placement of the catheter. The positioning of catheter 1 can be observed by a viewer due to the flexible tube 2 being translucent and the flexible core 9 being non-translucent. In addition, a viewer can view via X-ray or fluoroscopy the positioning of the catheter within the patient due to the flexible core 9 being radiopaque.

Because the flexible core 9 contains radiopaque material, therefore, the flexible tube 2 need not be radiopaque. The removal of the radiopaque material improves the mechanical and biostability properties of the flexible tube 2.

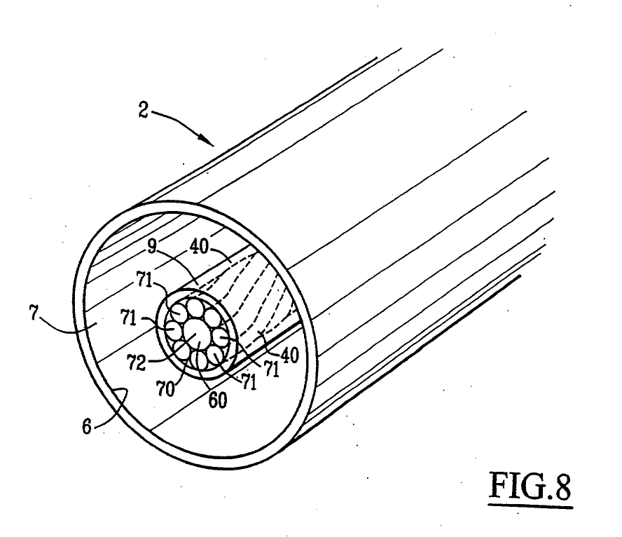

In another preferred embodiment, the flexible core 9 comprises a stranded flexible wire bundle 60, as shown in FIG. 8. Wire bundle 60 can comprise wires 70 embedded in polyurethane. Jacket 40 may cover wires 70 to prevent fraying of wires 70 and to provide structural support as may be desired. Wires 70 can comprise any suitable radiopaque material as 5 previously described or conductor wire. Those skilled in the art will recognize that various alloys can be used, including alloys comprising, but not limited to, platinum, iridium, silver, and nickel, e.g., such as MP35N. Further, wires 70 can comprise outer wires 71 and at least one inner wire 72. Still further, outer wires 71 of bundle 60 can be twisted if desired around a inner wire 72, as is shown in FIG. 8. Those skilled in the art will recognize that wire bundle 60 can have non-twisted wires or any combination of twisted and non-twisted wires, and/or any combination of outer wires and inner wires. Thus, those skilled in the art will recognize the myriad of possible flexible core constructions in accordance with the present invention.

In another preferred embodiment, the flexible core 9 could also be electrically conductive to allow for electrical stimulation at the distal end (i.e., the end remote from the electrical power source (not shown) for the electrical stimulation. More specifically, flexible core 9 can comprise conductor wire or cable.

Alternatively, the flexible core 9 could be hollow to function as a fluid return path for the purpose of sampling patient fluid and/or draining of patient fluid. In this alternative preferred embodiment, an opening is provided in flexible tube 2 at distal end 12 and a corresponding opening in flexible core 9 so that patient fluid can flow into flexible core 9 without entering passageway 11. This construction eliminates the need to interrupt drug delivery through passageway 11 to the patient and/or to flush passageway 11 to obtain a sample of patient fluid or to drain away patient fluid.

The outside diameter of the flexible tube 2 can preferably be quite small (e.g., about 0.030 inches or smaller) and as great as up to about 2 inches. The outside diameter of the flexible core 9 will always be smaller than the inside diameter of the flexible tube 2 and can preferably be up to about 1.5 inches. The diameters of the both the flexible core 9 and the lumen 7 can be varied to adjust dead space, stiffness and fluid flow properties as desired. In a more preferred embodiment for intrathecal drug delivery, the outside diameter of the flexible tube 2 will be about 0.050 inches or smaller.

As previously noted, the flexible tube 2 and flexible core 9 can be co-extruded. Co-extrusion will permit the catheter wall and/or core stiffness to be varied under controlled conditions as desired. The ability to control the stiffness of the catheter wall and/or core, will allow for the manufacture of catheters without resort to current methods like the Total Intermittent Extruded (TIE) technique of Putnam Plastics Corporation of Dayville, Conn., or joining techniques where two pieces of catheter are pushed and heated together to form a joint, and/or techniques of pulling and stretching the catheter to vary the thickness of the walls of the catheter. Those of skill in the art will recognize that, if desired, co-extrusion of flexible tube 2 and flexible core 9 can be performed in a manner that results in the flexible core 9 being attached to the inner wall 6 of flexible tube 2, as shown in FIG. 4.

One embodiment of the present invention is a method of preventing occlusion in a flexible tube, comprising placing a flexible core within a flexible tube, the flexible tube having an outer wall and an inner wall, the inner wall defining a lumen, the lumen extending longitudinally along the flexible tube and having a cross-sectional area, the flexible core within the lumen, substantially extending longitudinally along the flexible tube, the flexible core having a cross-sectional area smaller than the cross-sectional area of the lumen at any given longitudinal point along the flexible tube and the flexible core, the flexible tube and the flexible core defining a passageway, and which in combination provides structure resistant to occlusion of the passageway. Further, the cross-sectional area of the flexible tube may be substantially constant. Another embodiment of the present invention is a method of making an occlusion resistant medical catheter, comprising (a) co-extruding polyurethane over a heavy metal loaded polyurethane to comprise the flexible core; and (b) placing a flexible core within a flexible tube, the flexible tube having an outer wall and an inner wall, the inner wall defining a lumen, the lumen extending longitudinally along the flexible tube and having a cross-sectional area, the flexible core within the lumen, substantially extending longitudinally along the flexible tube, the flexible core having a cross-sectional area smaller than the cross-sectional area of the lumen at any given longitudinal point along the flexible tube and the flexible core, the flexible tube and the flexible core defining a passageway, and which in combination provides structure resistant to occlusion of the passageway. Further, the cross-sectional area of the flexible tube may be substantially constant.

In a preferred embodiment of the present invention, the distal end 12 will be made of a soft material with a slight stiffness to reduce and/or eliminate tissue damage to the patient. In a preferred embodiment of the present invention, the proximal end 13 will have sufficient stiffness adequate for the proper and easy insertion and placement of the catheter into the patient. In a preferred embodiment of the invention, the proximal end 13 will have a stiffness of up to about 40 times greater the stiffness of distal end 12, and more preferably about 10 times greater the stiffness of distal end 12. The catheter should have sufficient stiffness to implant in a patient, yet be of sufficient softness after implant so as to reduce or prevent irritation to patient tissue. Polyurethane and related copolymers are the types of materials that will begin to soften shortly after implant (e.g., about 5-10 minutes) and thus are preferred materials for the distal end 12. More specifically, polyurethane and related co-polymers will soften after implant due to local tissue temperature and moisture after implant so as to reduce and/or eliminate tissue damage to the patient. Those skilled in the art will recognize other similar materials that are useful for distal end 12.

Those skilled in the art will recognize that the preferred embodiments may be altered or amended without departing from the true spirit and scope of the invention, as defined in the accompanying claims. Thus, while various alterations and permutations of the invention are possible, the invention is to be limited only by the following claims and equivalents.

What is claimed:

1. A medical catheter comprising:
(a) a flexible tube having an outer wall and an inner wall, the inner wall defining a lumen, the lumen extending longitudinally along the flexible tube and having a cross-sectional area;
(b) a flexible core within the lumen, substantially extending longitudinally along the flexible tube, the flexible core having a cross-sectional area smaller than the cross-sectional area of the lumen at any given longitudinal point along the flexible tube and flexible core, the flexible tube and the flexible core defining a passageway having a cross-sectional area, and which in combination provides structure resistant to occlusion of the passageway, the flexible core having at least one end being free to move within the lumen.

2. A medical catheter comprising:
(a) a flexible tube having an outer wall and an inner wall, the inner wall defining a lumen, the lumen extending longitudinally along the flexible tube and having a cross-sectional area;
(b) a flexible core within the lumen, substantially extending longitudinally along the flexible tube, the flexible core having a cross-sectional area smaller than the cross-sectional area of the lumen at any given longitudinal point along the flexible tube and flexible core, the flexible tube and the flexible core defining a passageway having a cross-sectional area, and which in combination provides structure resistant to occlusion of the passageway, the flexible core being permanently attached to the inner wall of the flexible tube.

3. A medical catheter comprising:
(a) a flexible tube having an outer wall and an inner wall, the inner wall defining a lumen, the lumen extending longitudinally along the flexible tube and having a cross-sectional area;
(b) a flexible core within the lumen, substantially extending longitudinally along the flexible tube, the flexible core having a cross-sectional area smaller than the cross-sectional area of the lumen at any given longitudinal point along the flexible tube and flexible core, the flexible tube and the flexible core defining a passageway having a cross-sectional area, and which in combination provides structure resistant to occlusion of the passageway, the flexible core comprising a wire bundle.

4. The medical catheter of claim 1, wherein the flexible core is initially attached to the flexible tube, but is free to break away from the flexible tube at a point of attachment.

5. The medical catheter of claim 1, wherein the longitudinal cross-section of the flexible core is circular in shape.

6. The medical catheter of claim 2, wherein the longitudinal cross-section of the flexible core is circular in shape.

7. The medical catheter of claim 3, wherein the longitudinal cross-section of the flexible core is circular in shape.

8. The medical catheter of claim 1, wherein the longitudinal cross-section of the flexible core is non-circular in shape.

9. The medical catheter of claim 2, wherein the longitudinal cross-section of the flexible core is non-circular in shape.

10. The medical catheter of claim 3, wherein the longitudinal cross-section of the flexible core is non-circular in shape.

11. The medical catheter of claim 1, wherein the longitudinal cross-section of the passageway is circular in shape.

12. The medical catheter of claim 2, wherein the longitudinal cross-section of the passageway is circular in shape.

13. The medical catheter of claim 3, wherein the longitudinal cross-section of the passageway is circular in shape.

14. The medical catheter of claim 1, wherein the longitudinal cross-section of the passageway is non-circular in shape.

15. The medical catheter of claim 2, wherein the longitudinal cross-section of the passageway is non-circular in shape.

16. The medical catheter of claim 3, wherein the longitudinal cross-section of the passageway is non-circular in shape.

17. The medical catheter of claim 1, wherein the longitudinal cross-section of the flexible core has a different shape than the longitudinal cross-section of the lumen.

18. The medical catheter of claim 2, wherein the longitudinal cross-section of the flexible core has a different shape than the longitudinal cross-section of the lumen.

19. The medical catheter of claim 3, wherein the longitudinal cross-section of the flexible core has a different shape than the longitudinal cross-section of the lumen.

20. The medical catheter of claim 1, wherein the longitudinal cross-section of the flexible core is radiopaque.

21. The medical catheter of claim 2, wherein the longitudinal cross-section of the flexible core is radiopaque.

22. The medical catheter of claim 3, wherein the longitudinal cross-section of the flexible core is radiopaque.

23. The medical catheter of claim 20, wherein the flexible core has a first longitudinal portion having a cross-sectional shape or size different from a second longitudinal portion of the flexible core in series with the first longitudinal portion.

24. The medical catheter of claim 21, wherein the flexible core has a first longitudinal portion having a cross-sectional shape or size different from a second longitudinal portion of the flexible core in series with the first longitudinal portion.

25. The medical catheter of claim 22, wherein the flexible core has a first longitudinal portion having a cross-sectional shape or size different from a second longitudinal portion of the flexible core in series with the first longitudinal portion.

26. The medical catheter of claim 1, wherein the flexible core is made of a heavy metal and polyurethane.

27. The medical catheter of claim 2, wherein the flexible core is made of a heavy metal and polyurethane.

28. The medical catheter of claim 3, wherein the flexible core is made of a heavy metal and polyurethane.

29. The medical catheter of claim 26, wherein the heavy metal is selected from the group consisting of tantalum, tungsten, gold, platinum, iridium, silver, nickel, and alloys thereof.

30. The medical catheter of claim 27, wherein the heavy metal is selected from the group consisting of tantalum, tungsten, gold, platinum, iridium, silver, nickel, and alloys thereof.

31. The medical catheter of claim 28, wherein the heavy metal is selected from the group consisting of tantalum, tungsten, gold, platinum, iridium, silver, nickel, and alloys thereof.

32. The medical catheter of claim 1, wherein the flexible core contains up to about 95% by weight of a heavy metal.

33. The medical catheter of claim 2, wherein the flexible core contains up to about 95% by weight of a heavy metal.

34. The medical catheter of claim 3, wherein the flexible core contains up to about 95% by weight of a heavy metal.

35. The medical catheter of claim 1, wherein the flexible core contains up to about 80-85% by weight of a heavy metal.

36. The medical catheter of claim 2, wherein the flexible core contains up to about 80-85% by weight of a heavy metal.

37. The medical catheter of claim 3, wherein the flexible core contains up to about 80-85% by weight of a heavy metal.

38. The medical catheter of claim 1, wherein the flexible core has a jacket.

39. The medical catheter of claim 2, wherein the flexible core has a jacket.

40. The medical catheter of claim 3, wherein the flexible core has a jacket.

41. The medical catheter of claim 1, wherein the jacket of the flexible core comprises polyurethane resin.

42. The medical catheter of claim 2, wherein the jacket of the flexible core comprises polyurethane resin.

43. The medical catheter of claim 3, wherein the jacket of the flexible core comprises polyurethane resin.

44. The medical catheter of claim 1, wherein the flexible core has a first longitudinal portion having radiopaque material and a second longitudinal portion in series with the first longitudinal portion, wherein the second longitudinal portion does not contain radiopaque material.

45. The medical catheter of claim 2, wherein the flexible core has a first longitudinal portion having radiopaque material and a second longitudinal portion in series with the first longitudinal portion, wherein the second longitudinal portion does not contain radiopaque material.

46. The medical catheter of claim 3, wherein the flexible core has a first longitudinal portion having radiopaque material and a second longitudinal portion in series with the first longitudinal portion, wherein the second longitudinal portion does not contain radiopaque material.

47. The medical catheter of claim 1, wherein the flexible core is electrically conductive.

48. The medical catheter of claim 2, wherein the flexible core is electrically conductive.

49. The medical catheter of claim 3, wherein the flexible core is electrically conductive.

50. The medical catheter of claim 1, wherein the flexible core comprises a wire bundle.

51. The medical catheter of claim 2, wherein the flexible core comprises a wire bundle.

52. The medical catheter of claim 50, wherein the wire bundle comprises at least one twisted wire.

53. The medical catheter of claim 51, wherein the wire bundle core comprises at least one twisted wire.

54. The medical catheter of claim 3, wherein the wire bundle comprises at least one twisted wire.

55. The medical catheter of claim 52, wherein the wire bundle comprises outer wires twisted around at least one inner wire.

56. The medical catheter of claim 53, wherein the wire bundle comprises outer wires twisted around at least one inner wire.

57. The medical catheter of claim 3, wherein the wire bundle comprises outer wires twisted around at least one inner wire.

58. The medical catheter of claim 50, wherein the wire bundle is covered with a jacket.

59. The medical catheter of claim 51, wherein the wire bundle is covered with a jacket.

60. The medical catheter of claim 3, wherein the wire bundle is covered with a jacket.

61. The medical catheter of claim 58, wherein the jacket comprises polyurethane resin.

62. The medical catheter of claim 59, wherein the jacket comprises polyurethane resin.

63. The medical catheter of claim 60, wherein the jacket comprises polyurethane resin.

64. The medical catheter of claim 50, wherein the wire bundle comprises a heavy metal alloy.

65. The medical catheter of claim 51, wherein the wire bundle comprises a heavy metal alloy.

66. The medical catheter of claim 3, wherein the wire bundle comprises a heavy metal alloy.

67. The medical catheter of claim 64, wherein the heavy metal alloy is selected from the group consisting of tantalum, tungsten, gold, platinum, iridium, silver, and nickel alloys.

68. The medical catheter of claim 65, wherein the heavy metal alloy is selected from the group consisting of tantalum, tungsten, gold, platinum, iridium, silver, and nickel alloys.

69. The medical catheter of claim 66, wherein the heavy metal alloy is selected from the group consisting of tantalum, tungsten, gold, platinum, iridium, silver, and nickel alloys.

70. A method of assembly of a medical catheter comprising placing a flexible core within a flexible tube, the flexible tube having an outer wall and an inner wall, the inner wall defining a lumen, the lumen extending longitudinally along the flexible tube and having a cross-sectional area, the flexible core within the lumen, substantially extending longitudinally along the flexible tube, the flexible core comprising a heavy metal and polyurethane and having a cross-sectional area smaller than the cross-sectional area of the lumen at any given longitudinal point along the flexible tube and the flexible core, the flexible tube and the flexible core defining a passageway, the flexible core having at least one end that is free to move within the lumen, and which in combination provides structure resistant to occlusion of the passageway.

71. The method of claim 70 wherein the longitudinal cross-sectional area of the flexible core is substantially constant.

72. A method of removing fluid from a target site of a patient comprising:
(a) implanting into a patient a catheter, the catheter having a flexible tube having an outer wall and an inner wall and defining at least one opening at a target site within a patient, the at least one opening providing fluid transfer from outside the outer wall to inside the inner wall, the inner wall defining a lumen, the lumen extending longitudinally along the flexible tube and having a cross-sectional area, a flexible core within the lumen, substantially extending longitudinally along the flexible tube, the flexible core having a cross-sectional area smaller than the cross-sectional area of the lumen at any given longitudinal point along the flexible tube and flexible core, the flexible tube and the flexible core defining a passageway having a cross-sectional area, and which in combination provides structure resistant to occlusion of the passageway; and
(b) drawing fluid away from the target site of the patient.

73. A method of making an occlusion resistant medical catheter, comprising:
(a) co-extruding polyurethane over a heavy metal loaded polyurethane to comprise the flexible core; and
(b) placing a flexible core within a flexible tube, the flexible tube having an outer wall and an inner wall, the inner wall defining a lumen, the lumen extending longitudinally along the flexible tube and having a cross-sectional area, the flexible core within the lumen, substantially extending longitudinally along the flexible tube, the flexible core having a cross-sectional area smaller than the cross-sectional area of the lumen at any given longitudinal point along the flexible tube and the flexible core, the flexible tube and the flexible core defining a passageway, and which in combination provides structure resistant to occlusion of the passageway.

74. The method of claim 73 wherein the cross-sectional area of the flexible tube is substantially constant.

75. The method of claim 73 further comprising the step of co-extruding the flexible core and the flexible tube in a manner that results in the flexible core being attached to the inner wall of the flexible tube.

76. The method of claim 73 wherein step (a) comprises co-extruding polyurethane over a heavy metal selected from the group consisting of tantalum, tungsten, gold, platinum, iridium, silver, and nickel, and alloys thereof.

77. A method of assembly of a medical catheter comprising placing a flexible core within a flexible tube, the flexible tube having an outer wall and an inner wall, the inner wall defining a lumen, the lumen extending longitudinally along the flexible tube and having a cross-sectional area, the flexible core within the lumen, substantially extending longitudinally along the flexible tube, wherein the flexible core contains up to about 95% by weight of a heavy metal, the flexible core having a cross-sectional area smaller than the cross-sectional area of the lumen at any given longitudinal point along the flexible tube and the flexible core, the flexible tube and the flexible core defining a passageway, and which in combination provides structure resistant to occlusion of the passageway.

78. The method of claim 77 wherein the cross-sectional area of the flexible core is substantially constant.
